(19) United States
(12) Patent Application Publication
(10) Pub. No.: US 2019/0177770 A1
(43) Pub. Date: Jun. 13, 2019

(54) DETECTION OF NUCLEIC ACIDS FROM PLATELET ENRICHED PLASMA SAMPLES

(71) Applicant: Roche Molecular Systems, Inc., Pleasanton, CA (US)

(72) Inventors: Emma Brown, Danville, CA (US); Dwight Kuo, Castro Valley, CA (US); Chitra Manohar, San Ramon, CA (US); Priscilla Moonsamy, Pleasanton, CA (US); Sneha Nishtala, San Ramon, CA (US); Lori Steiner, Alameda, CA (US)

(21) Appl. No.: 16/213,354

(22) Filed: Dec. 7, 2018

Related U.S. Application Data
(60) Provisional application No. 62/596,253, filed on Dec. 8, 2017.

(51) Int. Cl. C12Q 1/6806; C12Q 1/686; C12Q 1/6886

(52) U.S. Cl. CPC C12Q 1/6806; C12Q 1/6886; C12Q 1/686

(57) ABSTRACT

Provided herein are methods and compositions for isolating and detecting nucleic acids from platelet-enriched plasma.

### CROSS-REFERENCE TO RELATED APPLICATIONS

This patent application claims priority to U.S. Application No. 62/596,253, filed Dec. 8, 2017, the disclosure of which is incorporated herein by reference in its entirety.

### BACKGROUND OF THE INVENTION

Cell-free nucleic acids (cfRNA and cfDNA) from patient blood provide a non-invasive tool for molecular diagnosis but the amount recovered is often a limiting factor. More recently, patient plasma is often the liquid biopsy of choice for cfRNA and cfDNA or circulating tumor DNA (ctDNA) to monitor disease treatment and often for initial diagnosis. Yield of cell-free nucleic acids is very limited from patient plasma, which is most often used as a non-invasive sample type. Invasive tissue biopsies are often not feasible for cancer patients, especially at later stages.

Extracellular vesicles (EVs) found in biofluids throughout the body are shed from cells (Yanez-Mo et al. (2015) J. Extracellular Vesicles 4:27066), and cancer cells release an abundance of EVs, including exosomes, which contain shed tumor RNA into, and are present at a higher level in biofluids from cancer patients (Brock et al. (2015) Translational Cancer Res. 4:280). EVs shed by tumor cells are a source of cell free nucleic acids which can provide insight on the tumor cell of origin and include cancer associated biomarkers. There is increasing evidence that not just EVs exosomes and microsomes in the range of 30-100 nm and 100 nm-1 um, respectively), but larger bodies carry nucleic acids from the cells they derive from. Platelets, which are typically about 1-5 um, are the second most abundant cell type in blood, ranging from 150,000-300,000 per microliter, and have also been shown to he a source of tumor derived nucleic acids (e.g., Nilsson et al. (2011) Blood 118:3680). Indeed, platelets found in the blood of cancer patients can also carry biomolecules transferred to them in a tumor environment Best et at (2015) Cancer Cell 28:666). Platelets found in cancer patient biofluids are thus referred to as tumor educated platelets (TEPs) and can be another useful source for biomarkers to detect and characterize cancer in a non-invasive manner.

### SUMMARY OF THE INVENTION

Provided herein are kits, assays, and methods for detecting nucleic acids in platelet enriched plasma (PEP). In some embodiments, provided are methods for detecting a target nucleic acid comprising: (a) obtaining a blood sample from a subject; (b) separating platelet-enriched plasma (PEP) from other blood components; (c) purifying nucleic acids from the PEP; and (d) detecting the target nucleic acid. In some embodiments, the target nucleic acid is RNA, e.g., miRNA or mRNA. In some embodiments, the target nucleic acid is DNA. In some embodiments, detecting is carried out by RT-PCR. In some embodiments, detecting is carried out by a hybridization assay. In some embodiments, detecting is carried out by PCR or next generation sequencing.

In some embodiments, separating PEP comprises centrifuging the blood sample to separate PEP from red and white blood cells and isolating the PEP into a separate vessel, e.g., by pipetting out PEP, or by pipetting out the red and white blood cells. In some embodiments, the centrifugation is carried out at 100-200 g. In some embodiments, the centrifugation is carried out at 120-360 g. In some embodiments, the centrifugation is carried out in separate steps, e.g., so that platelets are separated at about 300-400 g, and plasma is separated at about 1000-1800 g, and the two components combined to form PEP. In some embodiments, separating PEP comprises filtering (e.g., size filtration) the blood sample to separate PEP from red and white blood cells.

In some embodiments, the target nucleic acid is present at a higher or lower level in PEP from a subject with cancer than in PEP from a control (e.g., subject or population without cancer, or at lower risk of cancer). For example, the target nucleic acid can be an RNA (e.g., mRNA, splice variant, or miRNA) known to be over- or under-expressed in cancer samples. In some embodiments, the target nucleic acid is present in a variant form in PEP from a subject with cancer compared to PEP from a control (e.g., subject or population without cancer, or at lower risk of cancer). In some embodiments, the variant form is an insertion, deletion, substitution, and/or fusion variant, or a copy number variant.

In some embodiments, the subject is suspected of having cancer or has been diagnosed with cancer. In some embodiments, the cancer is selected from cancer of the adrenal gland, blood (e.g., lymphoma or leukemia), brain, breast, cervix, colon or colorectal region, esophagus, kidney, liver, lung, ovary, pancreas, prostate, stomach, or testes.

In some embodiments, the method further comprises detecting that a target nucleic acid is present at a higher or lower level in the sample from the subject than in a control sample, and correlating that result (higher or lower level) with potential therapeutic options for the subject, and creating a report of the potential therapeutic options for the subject. In some embodiments, the method further comprises recommending or treating the subject based on the report.

In some embodiments, the method further comprises detecting that a target nucleic acid is present in a variant form in the sample from the subject than in a control sample, and correlating that result (variant form) with potential therapeutic options for the subject, and creating a report of the potential therapeutic options for the subject. In some embodiments, the method further comprises recommending or treating the subject based on the report.

Further provided herein are kits for preparing PEP from a blood sample, and detecting, a target nucleic acid in the PEP. In some embodiments, the kit comprises a blood sample collection vessel, wherein the vessel can withstand centrifugation at 50-5000 g, e.g., 100-500 g or 100-1800 g. In some embodiments, the kit comprises a blood sample collection vessel, and a separate vessel that can withstand centrifugation at 50-5000 g, e.g., 100-500 g or 100-1800 g.

In some embodiments, the kit further comprises reagents for nucleic acid purification For example, the kit can include a lysis buffer (e.g., for disrupting platelet and EV membranes), nucleic add stabilizing reagents, reagents for nucleic acid binding (e.g., chromatography reagents or magnetic beads), and/or wash and elution buffers.

In, some embodiments, the kit further comprises reagents for detecting a target nucleic acid. For example, the kit can include reagents for nucleic acid amplification and detection (e.g. by RT-PCR or PCR). In some embodiments, target-specific reagents are included, e.g. primers and/or probes for particular target sequences. In some embodiments, the kit further comprises controls, e.g., for determination of the efficiency of nucleic acid separation, controls known to be positive for a given biomarker (target nucleic acid) or negative for a given biomarker.

In some embodiments, the kit further includes other consumables, such as vessels for nucleic acid purification and/or detection.

### DETAILED DESCRIPTION OF THE INVENTION

### I. Introduction

Platelets are a source of tumor or cancer associated biomarkers (e.g., nucleic acids and proteins) in blood. Blood fractions such as plasma also carry cancer associated biomarkers, e.g., in extracellular vesicles (EVs) shed from cancer cells. Plasma includes both circulating tumor DNA (ctDNA) and RNA, while platelets carry primarily RNA. Platelet enriched plasma (PEP) provides a rich source of biomarkers, and its use ensures that a broad range of biomarkers are captured from a patient sample. Tumor educated platelets (TEPs) carry biomarkers derived from tumor cells or from the tumor environment. EVs are formed by a different mechanism than platelets, and thus may carry a different subset of biomarkers.

The present disclosure provides methods for preparing PEP, and shows that platelets carry relatively more nucleic acids per blood volume than plasma. Use of PEP for detection assays is thus more likely to detect disease associated biomarkers present at very low concentrations than use of plasma alone.

EVs such as exosomes and microsomes are typically 30-1000 nm in size. Platelet size varies between individuals but is typically about 2 um. Red and white blood cells, on the other hand, are generally at least Burn in size, so can be easily separated from platelets and other EVs. Disclosed herein are methods for preparing PEP and nucleic acid extraction for use in detection assays.

### II. Definitions

The terms “platelet enriched plasma (PEP),” “platelet rich plasma,” “platelet dense plasma,” and like terms refer to a non-naturally occurring plasma sample from an individual with a higher concentration of platelets than is found in a plasma sample from the individual that is not enriched for platelets. Plasma refers to a blood sample from which red and white blood cells are removed, but clotting factors remain (unlike a serum sample). PEP can be prepared similar to plasma, e.g., by centrifugation, gravity filtration, or size-based filtration, to remove cellular blood components but retain platelets. In some embodiments, PEP is prepared by separately isolating plasma and platelets from a blood sample, and then recombining the plasma and platelets.

The terms “cell-free nucleic adds,” “cell-free RNA,” “cell-free DNA,” and like terms in the context of the present disclosure refers to a non tissue sample (e.g., liquid biopsy) from an individual that has been processed to largely remove cells. Examples of non-tissue samples include blood, urine, saliva, tears, mucus, etc. Platelets are non-nucleated, but are sometimes classified as cells. Unless noted otherwise herein, cell-free nucleic acids do not include those from PEP.

The term “biomarker” can refer to any detectable marker used to differentiate individual samples, e.g., cancer versus non-cancer samples. Biomarkers include modification (e.g., methylation of DNA, phosphorylation of protein), differential expression, and mutations or variants (e.g., single nucleotide variations, insertions, deletions, splice variants, and fusion variants). A biomarker can be detected in a DNA, RNA, and/or protein sample. PEP in particular is a rich source of RNA, and thus useful for detecting not just mutations, but miRNA, fusions, and variant expression levels.

The term “multiplex” refers to an assay in which more than one target is detected. The terms “receptacle,” “vessel,” “tube,” “well,” “chamber,” “microchamber,” etc. refer to a container that can hold reagents or an assay. If the receptacle is in a kit and holds reagents, or is being used for an amplification reaction, it can be closed or sealed to avoid contamination or evaporation. If the receptacle is being used for an assay, it can be open or accessible, at least during set up of the assay.

The terms “individually detected” or “individual detection,” referring to a marker gene or marker gene product, indicates that each marker in a multiplex reaction is detected. That is, each marker is associated with a different label (detected by a differently labeled probe).

The terms “nucleic acid,” “polynucleotide,” and “oligonucleotide” refer to polymers of nucleotides (e.g., ribonucleotides or deoxyribo-nucleotides) and includes naturally-occurring (e.g., adenosine, guanidine, cytosine, uracil and thymidine), and non-naturally occurring (human-modified) nucleic acids. The term is not limited by length (e.g., number of monomers) of the polymer. A nucleic acid may be single-stranded or double-stranded and will generally contain phosphodiester bonds, although in some cases, nucleotide analogs may have other linkages. Monomers are typically referred to as nucleotides. The term “non natural nucleotide” or “modified nucleotide” refers to a nucleotide that contains a modified nitrogenous base, sugar or phosphate group, or that incorporates a non-natural moiety in its structure. Examples of non-natural nucleotides include dideoxynucleotides, biotinylated, aminated, deaminated, alkylated, benzylated and fluorophor-labeled nucleotides.

The term “primer” refers to a short nucleic acid (an oligonucleotide) that acts as a point of initiation of polynucleotide strand synthesis by a nucleic acid polymerase under suitable conditions. Polynucleotide synthesis and amplification reactions typically include an appropriate buffer, dNTPs and/or rNTPs, and one or more optional cofactors, and are, carried out at a suitable temperature. A primer typically includes at least one target-hybridized region that is at least substantially complementary to the target sequence (e.g. having 0, 1, 2, or 3 mismatches). This region is typically about 8 to about 40 nucleotides in length, e.g., 12-25 nucleotides. A “primer pair” refers to a forward and reverse primer that are oriented in opposite directions relative to the target sequence, and that produce an amplification product in amplification conditions. In some embodiments, multiple primer pairs rely on a single common forward or reverse primer. For example, multiple allele-specific forward primers can be considered part of a primer pair with the same, common reverse primer, e.g., if the multiple alleles are in close proximity to each other.

As used herein, “probe” means any molecule that is capable of selectively binding to a specifically intended target biomolecule, for example, a nucleic acid sequence of interest that hybridizes to the probes. The probe is detectably labeled with at least one non-nucleotide moiety. In some embodiments, the probe is labeled with a fluorophore and quencher.

The words “complementary” or “complementarity” refer to the ability of a nucleic acid in a polynucleotide to form a base pair with another nucleic acid in a second polynucleotide. For example, the sequence A-G-T (A-G-U for RNA) is complementary to the sequence T-C-A (U-C-A for RNA). Complementarity may be partial, in which only some of the nucleic acids match according to base pairing, or complete, where all the nucleic acids match according to base pairing. A probe or primer is considered “specific for” a target sequence if it is at least partially complementary to the target sequence. Depending on the conditions, the degree of complementarity to the target sequence is typically higher for a shorter nucleic acid such as a primer (e.g., greater than 80%, 90%, 95%, or higher) than fora longer sequence.

The term “specifically amplifies” indicates that a primer set amplifies a target sequence more than non-target sequence at a statistically significant level. The term “specifically detects” indicates that a probe will detect a target sequence more than non-target sequence at a statistically significant level. As will be understood in the art, specific amplification and detection can be determined using a negative control, e.g., a sample that includes the same nucleic acids as the test sample, but not the target sequence or a sample lacking nucleic acids. For example, primers and probes that specifically amplify and detect a target sequence result in a Ct that is readily distinguishable from background (non-target sequence), e.g., a Ct that is at least 2, 3, 4, 5, 5-10, 10-20, or 10-30 cycles less than background. The term “allele-specific” PCR refers to amplification of a target sequence using primers that specifically amplify a particular allelic variant of the target sequence. Typically, the forward or reverse primer includes the exact complement of the allelic variant at that position.

The terms “identical” or “percent identity,” in the context of two or more nucleic acids, or two or more polypeptides, refer to two or more sequences or subsequences that are the same or have a specified percentage of nucleotides, or amino acids, that are the same (e.g., about 60% identity, e.g., at least any of 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity casts over a region comprising a sequence that is at least about 8-25 amino acids or nucleotides in length, or over a region that is 50-100 amino acids or nucleotides in length, or over the entire length of the reference sequence.

The terms “isolate,” “separate,” “purify,” and like terms are not intended to be absolute. For example, isolation of RNA does not require 100% of non-RNA molecules to be removed, and separation of platelets does not require removal of 100% of non-platelet blood components. One of skill in the art will recognize an acceptable level of purity for a given situation.

The term “kit” refers to any manufacture (e.g , a package or a container) including at least one reagent, such as a nucleic acid probe or probe pool or the like, for specifically amplifying, capturing, tagging/converting or detecting RNA or DNA as described herein.

The term “amplification conditions” refers to conditions in a nucleic acid amplification reaction (e.g, PCR amplification) that allow for hybridization and template-dependent extension of the primers. The term “amplicon” or “amplification product” refers to a nucleic add molecule that contains all or a fragment of the target nucleic acid sequence and that is formed as the product of in vitro amplification by any suitable amplification method. One of skill will understand that a forward and reverse primer (primer pair) defines the borders of an amplification product. The term “generate an amplification product” when applied to primers, indicates that the primers, under appropriate conditions (e.g., in the presence of a nucleotide polymerase and NTPs), will produce the defined amplification product. Various PCR conditions are described in PCR Strategies (Innis et al., 1995, Academic Press, San Diego, Calif.) at Chapter 14; PCR Protocols: A Guide. to Methods and Applications (Innis, et al., Academic Press, N.Y., 1990)

The term “amplification product” refers to the product of an amplification reaction. The amplification product includes the primers used to initiate each round of polynucleotide synthesis. An “amplicon” is the sequence targeted for amplification, and the term can also be used to refer to amplification product. The 5′ and 3′ borders of the amplicon are defined by the forward and reverse primers.

The terms “individual”, “subject”, and “patient?” are used interchangeably herein. The individual can be pre-diagnosis, post-diagnosis but pre-therapy, undergoing therapy, or post-therapy. In the context of the present disclosure, the individual is typically seeking medical care.

The term “sample” or “biological sample” refers to any composition containing or presumed to contain nucleic acid. The term includes purified or separated components of cells, tissues, or blood, e.g., DNA, RNA, proteins, cell-free portions, or cell lysates. The sample can be FFPET, e.g., from a tumor or metastatic lesion. The sample can also be from frozen or fresh tissue, or from a liquid sample, e.g., blood or a blood component (plasma or serum), urine, semen, saliva, sputum, mucus, semen, tear, lymph, cerebral spinal fluid, mouth/throat rinse, bronchial alveolar lavage, material washed from a swab, etc. Samples also may include constituents and components of in vitro cultures of cells obtained from an individual, including cell lines. The sample can also be partially processed from a sample directly obtained from an individual, e.g., cell lysate or blood depleted of red blood cells.

The term “obtaining a sample from an individual” means that a biological sample from the individual is provided for testing. The obtaining can be directly from the individual, or from a third party that directly obtained the sample from the individual.

The term “providing therapy for an individual” means that the therapy is prescribe& recommended, or made available to the individual. The therapy may be actually administered to the individual by a third party (e.g., an in-patient injection), or by the individual herself.

A “control” sample or value refers to a value that serves as a reference, usually a known reference, for comparison to a test sample or test conditions. For example, a test sample can be taken from a test condition, e.g., from an individual suspected of having cancer, and compared to samples from known conditions, e.g., from a cancer-free individual (negative control), or from an individual known to have cancer or a target sequence of interest (positive control). In the context of the present disclosure, the test sample is typically from a cancer patient, or a patient suspected of having cancer. A control can also represent an average value or a range gathered from a number of tests or results. A control can also be prepared for reaction conditions. For example, a control for the presence, quality, and/or quantity of nucleic acid (e.g., internal control) can include primers or probes that will detect a sequence known to be present in the sample (e.g., a housekeeping gene such as beta actin, beta globin, glyceraldehyde 3-phosphate dehydrogenase (GAPDH), ribosomal protein L37 and L38, PPIase, EIF3, eukaryotic translation elongation factor 2 (eEF2), DHFR, or succinate dehydrogenase). In some embodiments, the internal control can be a sequence from a region of the same gene that is not commonly variant (e.g., in a different exon). A known added polynucleotide, e.g., having a designated length, can also be added. An example of a negative control is one free of nucleic acids, or one including primers or probes specific for a sequence that would not be present in the sample, e.g., from a different species. One of skill will understand that the selection of controls will depend on the particular assay, e.g., so that the control is cell type and organism-appropriate. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “label,” “tag,” “detectable moiety,” and like terms refer to a composition detectable by spectroscopic, photochemical, biochemical, immunochemical, chemical, or other physical means. For example, useful labels include fluorescent dyes (fluorophores), luminescent agents, radioisotopes (e.g., ³²P, ³H), electron dense reagents, or an affinity-based moiety, a poly-A (interacts with poly-T) or poly-T tag (interacts with poly-A), a His tag (interacts with Ni), or a strepavidin tag (separable with biotin). One of skill will understand that a detectable label conjugated to a nucleic acid is not naturally occurring.

Unless defined otherwise, technical and scientific terms used herein have the same meaning as commonly understood by a person of ordinary skill in the art. See, e.g:, Lackie, DICTIONARY OF CELL AND MOLECULAR BIOLOGY, Elsevier (4th ed. 007); Sambrook et al., MOLECULAR CLONING, A LABORATORY MANUAL, Cold Springs Harbor Press (Cold Springs Harbor, N.Y. 1989). The term “a or “an” is intended to mean one or more.” The terms “comprise,” “comprises,” and “comprising,” when preceding the Cecitation of a step or an element, are intended to mean that the addition of further steps or elements is optional and not excluded.

### III. Nucleic Acid Samples

Samples for biomarker detection can be obtained from any source suspected of containing nucleic acid, e.g., tissue, skin, swab (e.g., buccal, vaginal), urine, saliva, etc. In the context of the present disclosure, the sample is obtained from blood or a blood fraction.

In a sample that includes cells, the cells can be separated out (e.g., using size-based filtration or centrifugation), thereby leaving cell free nucleic acids (cfNA), including nucleic acids in exosomes, microvesicles, viral particles, or those circulating freely. In some embodiments, platelets are also captured with the cell-free component to form platelet enriched plasma.

Blood can be processed by any of at least three different methods. In addition, the same blood sample can be used to obtain platelets, plasma, and PEP. Blood can be centrifuged at different speeds to enrich and obtain platelet, plasma, and/or PEP fractions. For example, centrifugation at about 120 g (e.g., 100-200 g) results in PEP, about 360 g (e.g., 250-450 g) results in platelets (RNA), and about 1500 g (e.g., 1000-1800 g) results in plasma (RNA and DNA),

Methods for isolating nucleic acids from biological samples are known, e.g., as described in Sambrook, and several kits are commercially available (e.g., High Pure RNA Isolation Kit, High Pure Viral Nucleic Acid Kit, and MagNA Pure LC Total Nucleic Acid Isolation Kit, DNA Isolation Kit for Cells and Tissues, DNA Isolation Kit for Mammalian Blood, High Pure FFPET DNA Isolation Kit, available from Roche). In the context of the presently disclosed methods, RNA is collected, though in some embodiments, the classifier can be used on previously prepared cDNA.

### IV. Cancer Associated Nucleic Acids

PEP is useful for detecting disease related biomarkers that are present in a blood sample from an individual suffering from or at risk of disease. In particular, blood components are known to carry cancer-associated biomarkers, e.g., in nucleic acids carried in the blood. Cell-free nucleic acids, e.g., DNA in serum, and plasma, are typically present in fragments averaging 166 nucleotides (e.g, 50-300 bp, see, e.g., Lo et al. (2016) Trends Genet. 32:360). RNA present in EVs and platelets exhibit a size range from intact mRNA species to miRNAs that range in size from about 18-27 nucleotides (e.g., Lin et al., (2017) Annual Rev Cancer Biol. 1:163). PEP thus contains nucleic acids (RNA and DNA) from plasma, and RNA from platelets, constituting a superior liquid biopsy enriched for a broad range of biomarkers. Platelets are a more enriched source of miRNA than plasma, however plasma includes ctDNA not found in platelets. Cancer associated biomarkers include insertion, deletion, and substitution mutations, fusion variations, splice variants, miRNAs (e.g., presence and/or levels), differential expression levels (e.g., compared to a non-cancer sample), and copy number variations.

The most comprehensive source for cancer associated mutations is the COSMIC (Catalog of Somatic Mutations in Cancer) database, available at the website cancer.sanger.ac.uk (Version 81 May 2017). PEP can be used as a sample source to detect any mutation listed in the database, as appropriate for the individual providing the sample. The COSMIC database categorizes biomarkers in a number of ways, including tissue of origin, therapeutic effect, and signaling pathway. For miRNA, miRbase (the database found at mirbase.org) provides a comprehensive data source. Thus, a medical provider can select markers associated with liver cancer for a patient with liver cancer, and later interrogate the database for mutations associated with drug resistance if the patient does not respond, or relapses, in response to first line therapy. PEP can be used as a sample source to detect any mutation listed in the database, as appropriate for the individual providing the sample. PEP is especially advantageous for monitoring because it is obtained in a relatively non-invasive manner, and provides a relatively concentrated source of cancer associated biomarkers.

In some embodiments, the biomarker is an insertion or deletion mutation. Examples of indel mutations that can be detected using the disclosed PEP sample source include but are not limited to: MET exon 14 deletion or VHL deletion.

In some embodiments, the biomarker is a substitution mutation (e.g., missense, nonsense, SNP). Examples of genes that include cancer-associated mutations that can be detected using the disclosed PEP sample source include but are not limited to: EGFR, BRAF, NRAS, KRAS, ABL1, ADAMTS5, ALK, APC, ARAF, ARID1A, ATM, ATP2B4, B2M, BCL2, BCL6, BCL7A, BTG1, CARD11, CCND3, CD58, CD274 (PDL1), CD798, CDH9, CDKN2A, CIITA, CNNB1, CNTNAP2, CPXCR1, CREBBP, CSMD3, CTNNB1, DCDC1, DDR2, DUSP22, EML4, EP300, EPHA6, ERBB2, ERBB3, ESR1, EZH2, FBXW7, FGFR1, FGFR2, FOXO1, FOXP1, GATA3, GNA13, GNAS, GRK7, GRM8, HCN1, HIST1H1B, HIST1H1C, HIST1H1E, IDH1, IKZF3, IRF4, ITPKB, JAK2, JAK3, KCNB2, KDM4C, KIT, LRIG3, LRRIQ3, MAP2K1, MEF2B, MET, MLH1, MSN, MYC, MYD88, NOTCH1, NOTCH2, NRXN1, PDL2, PGM5, PIK3CA, PIM1, PNPLA1, POM121L12, POU2F2, PTEN, PTPN1, PTPN11, PTPN6, PTPRD, RAF1, RB1, REG3A, REL, ROBO2, ROS1, S1PR2, SATB2, SGK1, SLC34A2, SLPI, SMAD4, SMARCB1, SOCS1, SPTA1, ST6GAL2, STAT6, SYT4, TBL1XR1, TNFAIP3, TNFRSF14, TNR, TP53, TRHDE, TR1M58, UNC5C, VHL, XPO1, ZIC4, and ZNF598.

In some embodiments, the cancer associated biomarker(s) includes at least one fusion variant. Examples of fusion variants that can be detected include those involving tyrosine kinases such as ALK, RET, ROS, NTRK (neurotrophic tyrosine receptor kinase), BRAE, ABL, and FGFR (fibroblast growth factor receptor). Particular examples of fusion variants that can be detected using the presently disclosed PEP sample source include but are not limited to: EML4-ALK, EML4-CCDC142, CCDC142-ALK, KIF5B-ALK, HIP1-ALK, KLC1-ALK, TFG-ALK, KIF5B-RET, CCD6-RET, NCOA4-RET, TRIM33-RET, ERCI-RET, BCR-ABL, CD74-RABGAP1L, RAD51AP2-ALK, EML-AKAP13, DCTN1-ALK, EML4-RABGAP1L, CD74-ROS1, STRN-ALK, MYO7A-ALK, EML4-LBH, EML-CUX1, FGFR3-TACC3, C11orf95-RELA, DNAJB1-PRKACA, TMPRSS2-ERG, PML-RARA, EGFR-SEPT14, RPS6KB1-VMP1, ETV6-NTRK3, SND1-BRAF, ETV6-ROSI, EML-AFF3, MLL-MLLT10, MLL-ELL, EHMT1-GRIN1I, NSD1-ZFN346, PPP1CB-PLB1, KDM2A-RHOD, NSD1-NUP98, and MLL-MLLT4 (see, e.g., Yoshihara et al. (Dec. 15, 2014) Oncogene).

In sonic embodiments, the cancer associated biomarker(s) includes at least one copy number variation (CNV). Examples of CNVs that can be detected using the presently disclosed PEP sample source include but are not limited to: AKT1, AR, ATM, C6, CCND1, CCND2, CNBD1, CWF19L2, DCDC2, DIO2, ERBB2, ERBB3, EPHX4, ESR1, EXOC4, FERD3L, FGFR1, GNA11, GRM8, IDH2, INSL5, KRAS, KIF5B, KIT, MAP2K1, MYD88, NPM1, PDGFRB, SLC17A8, SLC5A10, SLP1, TNR, and TP53.

Detection of a cancer associated biomarker can be used to diagnose the cancer associated with the biomarker, predict the likelihood of developing the cancer associated with the biomarker, select an appropriate treatment for a patient, monitor therapeutic progress of a patient undergoing cancer therapy, provide a prognosis for a cancer patient.

In some embodiments, targeted therapy is prescribed, provided, or administered to the patient based on the presence or absence of a cancer-associated biomarker. Several drugs are targeted for patients with specific biomarker profiles. For example, patients with an EGFR mutation can receive targeted therapy selected from, e.g., afatinib, cetuximab, dacomitinib, erlotinib, gefitinib, HG-5-88-01, lapatinib, osimertinib, and pelitinib. Patients with a mutation in VEGFR, KIT, or PDGFR can receive targeted therapy selected from, e.g., amuvatinib, axitinib, cabozatinib, imatinib, motesanib, masitinib, ponatinib, pazopanib, and sorafenib. New targeted therapies are being developed to address a number of specific mutations, thus one of skill in the art will be in the best position to select a targeted therapy for an individual at the time.

In addition, patients can benefit from standard chemotherapy. Thus, in some embodiments, chemotherapy is prescribed, provided, or administered to the patient based on the presence or absence of a cancer-associated biomarker. This can include CHOP (cyclophosphamide; doxorubicin; vincristine; and prednisolone) or R-CHOP, which further includes rituximab and/or etoposide. The cocktail can be administered periodically for a set period of time, or until reduction in tumor size and/or symptoms are detected. For example, the CHOP or R-CHOP can be administered every 2 or 3 weeks.

Regardless of which treatment is selected, it typically begins with a low dose so that side effects can be determined, and the dose increased, e.g., until side effects appear or within the patient's tolerance, or until clinical benefit is observed.

### V. Amplification and Detection

A nucleic acid sample can be used for detection and quantification, e.g., using nucleic acid amplification, e.g., using any primer-dependent method. For detection of a biomarker in an RNA sample, a preliminary reverse transcription step is carried out (also referred to as RT-PCR, not to be confused with real time PCR). See, e.g., Hierro et al. (2006) 72:7148. The term “gRT-PCR” as used herein refers to reverse transcription and quantitative PCR. Both reactions can be carried out in a single tube without interruption, e.g., to add reagents. For example, a polyT primer can be used to reverse transcribe all mRNAs in a sample with a polyA tail, random oligonucleotides can be used, or a primer can be designed that is specific for a particular target transcript that will be reverse transcribed into cDNA. The cDNA, or DNA from the sample, can form the initial template to be used for quantitative amplification (real time or quantitative PCR, i.e., RTPCR or qPCR). qPCR allows for reliable detection and measurement of products generated during each cycle of the PCR process. Such techniques are well known in the art, and kits and reagents are commercially available, e.g., from Roche Molecular Systems, Life Technologies, Bio-Rad, etc. See, e.g., Pfaffl (2010) Methods: The ongoing evolution of qPCR vol. 50.

A separate reverse transcriptase and thermostable DNA polymerase can be used, e.g., in a two-step (reverse transcription followed by addition of DNA polymerase and amplification) or combined reaction (with both enzymes added at once). in some embodiments, the target nucleic acid is amplified with a thermostable polymerase with both reverse transcriptase activity and DNA template-dependent activity. Exemplary enzymes include Tth DNA polymerase, the C. therm Polymerase system, and those disclosed in US20140170730 and US20140051126.

Probes for use as described herein can be labeled with a fluorophore and optionally a quencher (e.g., TaqMan, LightCycler, Molecular Beacon, Scorpion, or Dual Labeled probes). Appropriate fluorophores include but are not limited to PAM, JOE, TET, Cal Fluor Gold 540, HEX, VIC, Cal Fluor Orang 560, TAMRA, Cyanine 3, Quasar 570, Cal Fluor Red 390, Rox, Texas Red, Cyanine 5, Quasar 670, and Cyanine 53. Appropriate quenchers include but are not limited to TAMRA (for FAM, JOE, and TET), DABCYL, and BHQ1-3.

Detection devices are known in the art and can be selected as appropriate for the selected labels. Detection devices appropriate for quantitative PCR include the cobas® and Light Cycler® systems (Roche), PRISM 7000 and 7300 real-time PCR systems (Applied Biosystems), etc. Six-channel detection is available on the CFX96 Real Time PCR Detection System (Bio-Rad) and Rotorgene Q (Qiagen), allowing for a higher degree of multiplexing.

For PCR detection, results can be expressed in terms of a threshold cycle (abbreviated as Ct, and in some instances Cq or Cp). A lower Ct value reflects the rapid achievement of a predetermined threshold level, e.g., because of higher target nucleic acid concentration or a more efficient amplification. A higher Ct value may reflect lower target nucleic acid concentration, or inefficient or inhibited amplification. The threshold cycle is generally selected to be in the linear range of amplification for a given target. In some embodiments, the Ct is set as the cycle at which the growth signal exceeds a pre-defined threshold line, e.g., in relation to the baseline, or by determining the maximum of the second derivation of the growth curve. Determination of Ct is known in the art, and described, e.g., in U.S. Pat. No. 7,363,168.

In some embodiments, digital PCR (dPCR) can be used to detect a cancer associated biomarker in PEP. For example, digital droplet PCR (ddPCR) can be used to determine absolute measurement of a target nucleic acid in a sample, even at very low concentrations. The dPCR method comprises the steps of digital dilution or droplet generation, PCR amplification, detection and (optionally) analysis. The partitioning step comprises generation of a plurality of individual reaction volumes (e.g., droplets) each containing reagents necessary to perform nucleic acid amplification. The PCR amplification step comprises subjecting the partitioned volumes to thermocycling conditions suitable for amplification of the nucleic acid targets to generate amplicons. Detection comprises identification of those partitioned volumes that contain and do not contain amplicons. The analysis step comprises a quantitation that yields e.g., concentration, absolute amount or relative amount (as compared to another target) of the target nucleic acid in the sample. Commercially available dPCR systems are available, e.g., from Bio-Rad, RainDance, and ThermoFisher. Descriptions of dPCR can be found, e.g., in US20140242582; Kuypers et al. (2017) J Clin Microbiol 55:1621; and Whale et al. (2016) Biomol Detect Quantif 10:15.

In some embodiments, the disease-associated biomarker is detected using sequencing, e.g., massively parallel sequencing (MPS) or next-generation sequencing (NGS). Next-generation sequencing methods clonally propagate millions of single DNA molecules in parallel. Each clonal population is then individually sequenced. NGS methods include sequencing by synthesis (e.g., Illumina), nanopore sequencing (e.g., Oxford Nanopore Technologies), single molecule real-time sequencing (e.g., Pacific Biosciences), ion semiconductor based sequencing (Ion Torrent), and pyrosequencing (454/Roche). Cell-free nucleic acids are present in short fragments, e.g., about 50-200 bp, thus read length limitations of the sequencing method is unlikely to be an issue. In some embodiments, the sequencing method comprises an optional target enrichment step, e.g., an amplification step. In other embodiments, other target enrichment methods are used, e.g., library-based or probe-based methods of target enrichment (e.g., U.S. Pat. No. 7,867,703 or U.S. Pat. No. 8,383,338). NGS methods are described, e.g., in Xu, Next Generation Sequencing: Current Technologies and Applications, Caister Acad. Press 2014; Ma et al. (2017) Biomicrofluidics 11:021501; Kelly (2017) Semin Oncol Nurs 33:208; and Serrati et al. (2016) Onco Targets Ther 9:7355.

In some embodiments, the disease-associated biomarker is detected using a hybridization method such as array analysis. Arrays typically utilize microchips with thousands of addressable locations that bind to specific target nucleic acids. Commercially available array systems are available from Affymetrix. For example, the GeneChip system can be used to detect both expression levels and sequence information. Details about and applications of microarray analysis are described e.g., in Bumgarner (2013) Curr Protoc Mol Biol 101: 22.1.

### VI. Kits

Provided herein are kits for carrying out separation of platelet-enriched plasma (PEP) from other blood components.

In some embodiments, the kit comprises a blood collection vessel (e.g., tube, vial, multi-well plate or multi-vessel cartridge). In some embodiments, the collection vessel is sufficiently durable to withstand centrifugation, e.g., at 50-5000×g, 100-1000×g.

In some embodiments, the blood collection vessel has a component for size filtration, e.g., a 1, 2, 3, 4, 5, 2-4, or 3-5 micron filter, to separate platelets and extracellular vesicles from cellular material. In some embodiments, the size filtration component is provided separately for insertion into a sample vessel, e.g., the blood collection vessel or a separate sample vessel. In some embodiments, the size filtration component is a spin column. In some embodiments, the size filtration component is a passive filter.

In some embodiments, the kit includes reagents and/or components for nucleic acid purification. For example, the kit can include, a lysis buffer (e.g., comprising detergent, chaotropic agents, buffering agents, etc.), enzymes or reagents for denaturing proteins or other undesired materials in the sample (e.g., proteinase K, DNase), enzymes to preserve nucleic acids DNase and/or RNase inhibitors). In some embodiments, the kit includes components for nucleic acid separation, e.g., solid or semi-solid matrices such as chromatography matrix, magnetic beads, magnetic glass beads, glass fibers, silica filters, etc. In some embodiments, the kit includes wash and/or elution buffers for purification and release of nucleic acids from the solid or semi-solid matrix. For example, the kit can include components from MagNA Pure LC Total Nucleic Acid Isolation Kit, DNA Isolation Kit for Mammalian Blood, High Pure or MagNA Pure RNA Isolation Kits (Roche), DNeasy or RNeasy Kits (Qiagen), PureLink DNA or RNA Isolation Kits (Thermo Fisher), etc.

In some embodiments, the kit includes reagents for detection of particular target nucleic acids, e.g., target nucleic acids associated with cancer. For example, the kit can include oligonucleotides that specifically bind to cancer-associated biomarkers such as mutations or sequences, known to have copy number variations in cancer. In some embodiments, the detection reagents are for RT-PCR, qRT-PCR, qPCR, dPCR, sequencing (Sanger or NGS).

The kit can further include reagents for amplification, e.g., reverse transcriptase, DNA polymerase, dNTPs, buffers, and/or other elements (e.g., cofactors or aptamers) appropriate for reverse transcription and/or amplification. Typically, the reagent mixture(s) is concentrated, so that an aliquot is added to the final reaction volume, along with sample (e.g., RNA or DNA), enzymes, and/or water. In some embodiments, the kit further comprises reverse transcriptase (or an enzyme with reverse transcriptase activity), and/or DNA polymerase (e.g., thermostable DNA polymerase such as Taq, ZO5, and derivatives thereof).

In some embodiments, the kit further includes at least one control sample, e.g., nucleic acids from non-cancer sample (or pooled samples), or from a sample known to carry a target sequence (or pooled samples). In some embodiments, the kit includes a negative control, e.g., lacking nucleic acids, or lacking mutant nucleic acids. In some embodiments, the kit further includes consumables, e.g., plates or tubes for nucleic acid preparation, tubes for sample collection, etc. In some embodiments, the kit further includes instructions for use, reference to a website, or software.

### VII. Examples

### Assessment of Cancer Associated Biomarker HER2 Levels in Platelets and Plasma

We selected the HER2 oncogene as a cancer associated biomarker to compare biomarker levels in plasma and platelets from the same volume of blood. Two, 5 ml blood samples were taken from each of three breast cancer patients and processed separately.

Platelet enriched plasma was prepared in a swinging bucket Eppendorf 5810R centrifuge at 120×g to pellet the red blood cells. The PEP was removed, carefully avoiding the white blood cell layer, and transferred to a new tube The PEP was then spun at 360×g to pellet the platelets. Platelets were washed in PBS +0.4% EDTA and then collected in 100 ul RNAlater. Platelets were either frozen at −80 C. or extracted using a manual plasma cfRNA sample preparation method based on the Roche High Pure Kit. Plasma was prepared by centrifugation at 1500×g, then extracted using a manual plasma cfRNA sample preparation method.

For both sample types, the eluate was analyzed to determine RIN score (RNA integrity). The values indicated that RNA quality did not vary greatly between platelets and plasma. Nucleic acids from an equivalent of 1 ml of blood (20 ul) from each of the samples were used to run duplicate assays for both a housekeeping gene (SDH) and HER2.

Expression of both the housekeeping gene and HER2 was detectable significantly earlier in the platelet samples compared to the plasma samples (ranging from 2-4 Ct, respectively). A difference of 3.3 Ct is equivalent to a 10-fold difference. Thus, the results indicate that platelets have about 6-9 fold more nucleic acid recovery than plasma. For HER2 expression in HER2+ breast cancer patients, a 1.4 to 4.3 fold increase in Ct from platelets vs plasma equates to a 4-13 fold higher RNA recovery. This is a significant improvement, allowing for better dynamic range determination.

Platelets and extracellular vesicles found in plasma are formed through different cellular processes. Platelets are typically generated by megakaryocytes in the bone marrow, but can pick up extracellular vesicles in the blood (Nilsson et al. (2011) Blood 118: 3680). Platelets are particularly rich in RNAs, including miRNA, while plasma also includes DNA, including ctDNA. Extracellular vesicles found in plasma can be shed by nearly any cell. Biomarkers found in each can add unique information about the condition of a patient, such as the origin or etiology of cancer in the patient.

Assessment of Cancer Associated miRNAs in PEP

MicroRNA (miRNA) can regulate mRNA expression, function as an oncogene or tumor suppressor, or be involved in regulation of metastasis. miRNA is thus an attractive model for targeted therapeutics, as it can serve for diagnosis or as a therapeutic itself Particular miRNAs are differentially present at various levels in certain cancers, and thus also serve as an attractive diagnostic tool. Another advantage is that miRNA is relatively stable.

We sought to detect and compare levels of miRNAs in plasma, PEP, and platelet samples from prostate cancer patients. Current tests for prostate cancer focus on prostate specific antigen (PSA), which is neither specific nor sensitive, and leads to overtreatment. Several miRNAs have been implicated (either upregulated or downregulated) in prostate cancer, so we selected 1.1 for detection by qRT-PCR. These included miR21 -5p, Let7i-5p, miR20a-5p, miR30c-5p, miR200b-3p, miR141-3p, miR375-3p, miR145-5p, miR221-5p, miR30c-5p, and standard control 130b-3p. Additional miRNAs were detected by NGS, including among others miR100-5p, miR6749-5p, miR155-5p, miR31-5p, miR99a-5p, miR7107-5p, miR218-5p, miR4632-3p mi R4433a-3p, miR335-5p, miR3613-5p, and miR370-3p. Results from qRT-PCR and NGS were compared and were largely in agreement.

Prior to obtaining samples from prostate cancer patients, we evaluated expression in prostate cancer cell lines. PC3, an androgen resistant cell-line derived from bone metastasis, is invasive and metastatic. LnCap was derived from a lymph node metastasis, is androgen sensitive, and can be induced to undergo androgen independent progression to become metastatic. Good read alignments and read size distributions were obtained for our reference NGS method for both cell-lines. Levels of the selected miRNAs detected by qRT-PCR versus NGS read counts generally yielded data that trended in the same direction and matched for highly expressed miRNAs. Both cell lines had high expression by qRT-PCR of miR21-5p, Let7i-5p, miR20a-5p, miR30c-5p, and miR200b-3p, and LnCap also expressed miR141-3p highly.

Blood was obtained from four prostate cancer patients and a healthy individual. Each blood sample was processed to yield platelets, PEP, and plasma.

In general, platelets give the highest expression, followed by PER then plasma, though there are some exceptions. Many of these miRNAs show significantly higher expression (e.g., miR200b-3p in platelets) or lower expression (e.g., miR145-5p) relative to a cutoff. The assays revealed platelets to be a particularly rich source of miRNA, while it is relatively rare in plasma. The following miRNAs were more highly expressed in all patient platelets vs a healthy donor sample: miR200b-3p, miR30c-5p, miR375-3p, and Let 7i-5p. miR145-5p was downregulated in all patient platelets.

When results from NGS methods were correlated and compared by LogFc (for a single patient vs a control) and p value (for more than one patient vs a control), the following miRNAs were common to both, and therefore considered the most relevant prostate cancer biornarkers: miR4433a-3p, miR335-5p, miR3613-5p, and mRiR370-3p.

The results show that plasma and platelets contribute different amounts of different biomarkers (e.g., ctDNA vs RNA, different miRNAs, different RNA fragments, etc.). PEP can therefore be used to detect a broader range of biomarkers, and minimize the amount of sample required from a given patient. Moreover, given that miRNAs are involved in many cellular processes, not just cancer (sec, e.g., Ardekani and Nacini (2010) Avicenna J. Med. Biotechnol. 2:161), PEP can be used as a source of miRNA for detecting or monitoring non-cancerous conditions.

One of skill will understand that different configurations can be made, depending on the variants of interest and selected detection method.

While the invention has been described in detail With reference to specific examples, it will be apparent to one skilled in the art that various modifications can be made within the scope of this invention. Thus the scope of the invention should not be limited by the examples described herein. All patents, publications, websites, Genbank (or other database) entries disclosed herein are incorporated by reference in their entireties.

1. A method of detecting a target nucleic acid comprising:
a) obtaining a blood sample from a subject;
b) separating platelet-enriched plasma (PEP) from other blood components;
c) purifying nucleic acids from the PEP; and
d) detecting the target nucleic acid,
wherein detection is carried out by PCR, next generation sequencing (NGS), or hybridization, and wherein the target nucleic acid is associated with cancer.

2. The method of claim 1, wherein the target nucleic acid is present at a higher or lower level in PEP from a subject with cancer than in PEP from a subject without cancer.

3. The method of claim 1, wherein the target nucleic acid is present in a variant form, in PEP from a subject with cancer compared to PEP from a subject without cancer.

4. The method of claim 3, wherein the variant is an insertion, deletion, substitution, and/or fusion variant.

5. The method of any one of the preceding claims, wherein the nucleic acid purified in step c) is RNA.

6. The method of claim 5, wherein the detecting is carried out by reverse transcriptase PCR.

7. The method of claim 5, wherein the target nucleic acid is an miRNA.

8. The method of any one of claims 1-4, wherein the nucleic acid purified in step c) is DNA.

9. The method of any one of the preceding claims, wherein separating PEP comprises centrifuging the blood sample to separate PEP from red and white blood cells and isolating the PEP in a separate vessel.

10. The method of claim 9, wherein the centrifuging is carried out at 120-360 times gravity.

11. The method of claim 1, wherein separating PEP comprises filtering the blood sample to separate PEP from red and white blood cells.
